(19) United States
(12) Plant Patent Application Publication
(10) Pub. No.: US 2005/0193452 P1
(43) Pub. Date: Sep. 1, 2005

(54) Variety of Daylily plant named 'BERRUB'

(50) HEMEROCALLIS HYBRID; DAYLILY

(76) Inventor: Leo Berbee, AD Lisse (NL)

Correspondence Address: FULWIDER PATTON LEE & UTECHT, LLP, 200 OCEANGATE, SUITE 1550, LONG BEACH, CA, 90802, US

(21) Appl. No.: 10/789,332

(22) Filed: Feb. 27, 2004

(51) Int. Cl. A01H 5/00

(52) U.S. Cl. PLT/312

(57) ABSTRACT

A new and distinct cultivar of Daylily plant named ‘BERRUB’ primarily characterized by its deep rich red flowers.

### BACKGROUND OF THE NEW PLANT

The present invention relates to a new and distinct cultivar of Daylily, botanically known as Daylily hemerocallis and further known by the varietal name ‘BERRUB’. This new variety is also sold under the trade name RUBY STELLA (Breeder's Ref. No. D 92-54). The new variety was discovered in a seedling selection program by the inventor in Lisse, The Netherlands in 1992. The parental cultivar are unknown. The cultivar was isolated by the inventor, Leo Barbee, at Lisse, The Netherlands, by seedling selection primarily on the basis of ornamental appeal, particularly flower color.

Following selection of the seedling the cultivar was propagated vegetatively from clump divisions at Lisse, The Netherlands in 1992. Continued observation of the vegetative divisions resulting from asexual reproduction of clump divisions taken at Lisse, The Netherlands have confirmed that the distinguishing features of this new cultivar came true, remain stable and are retained through successive propagation.

### SUMMARY OF THE INVENTION

Plants of the cultivar ‘BERRUB’ have not been observed under all possible environmental and cultural conditions. The phenotype may vary somewhat with variations in environment such as temperature, light intensity, length of day, irrigation amount and frequency, and/or fertilizer rate without, however, any variance in genotype.

The following traits have been repeatedly observed and are determined to be among the unique characteristics of ‘BERRUB’. These characteristics distinguish ‘BERRUB’ as a new and distinct cultivar:

1. Distinctive rich burgundy red flower color.

Compared to other plants of the Daylily genus, the new Daylily has a distinctive generally trumpet shaped flower with a rich burgundy red flower.

### BRIEF DESCRIPTION OF THE DRAWINGS

The invention is depicted in the accompanying colored photographs in which:

FIG. 1 shows a close up of the flower and surrounding foliage; and

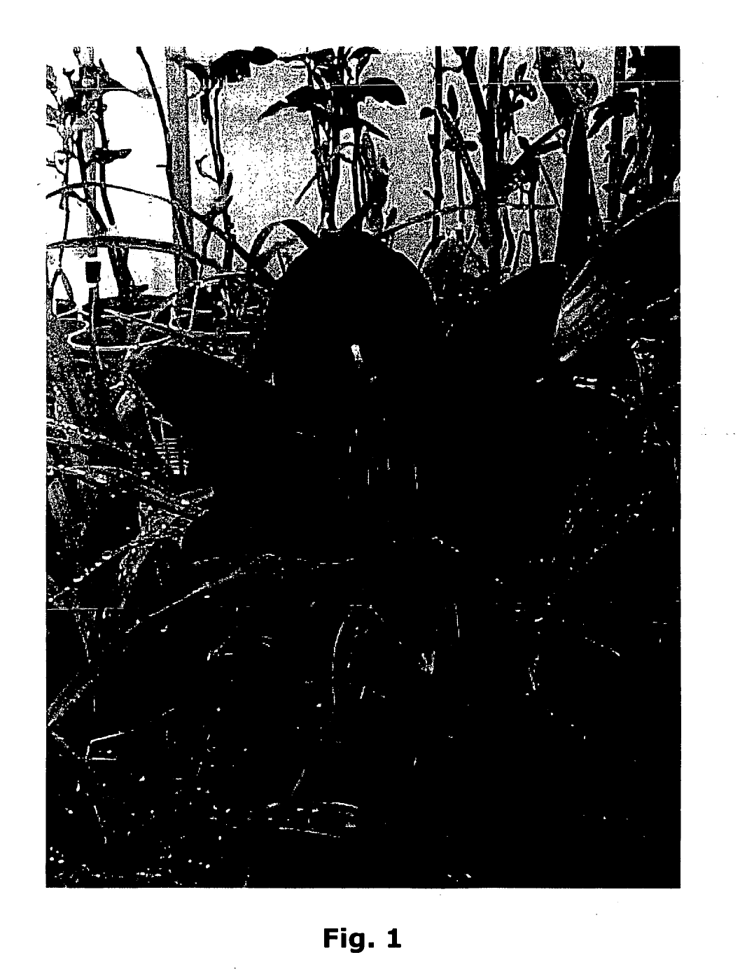

FIG. 2 shows the plant potted in a container.

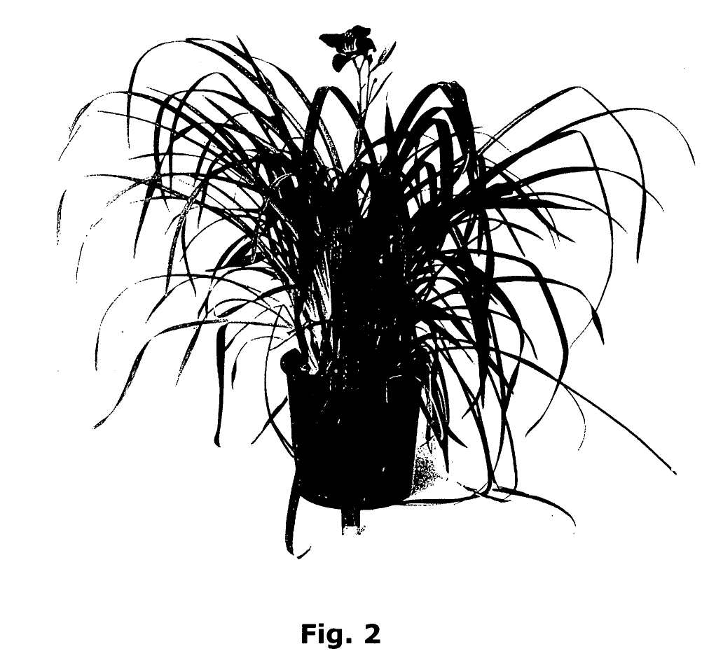

The photographs depict the overall appearance of the new Daylily, showing the colors as true as it is reasonably possible to obtain in colored reproductions of this type. Colors in the photographs may differ slightly from the color values cited in the Detailed Botanical Description which more accurately describe the actual colors of the new Daylily.

### DETAILED BOTANICAL DESCRIPTION OF THE NEW PLANT

The following detailed description sets forth characteristics of the new cultivar. The data which defines each characteristic was collected from asexual reproductions of the plant grown in an open greenhouse with irrigation provided by drip feeding in a hydroponic system, and carried out by the breeder at Clyde, Victoria, Australia, approximately 38 degrees, 9 minutes South latitude at 16 m elevation. The plant histories were taken on 3 plants in liners, potted into 150 mm and 200 mm pots in co-co coir starting on approximately April 2003 and running through January 2004.

In the following table of plant characteristics, color references are made to The Royal Horticultural Society of London Colour Chart, 1995 Edition. The color values were determined on plant material grown in an open greenhouse at Lisse, The Netherlands.

| | ‘BERRUB’ |
|---|---|
| The Plant | |
| Plant Growth Rate | Moderate |
| Length of Flowering Season | January to May |
| Time of Flowering Season | Mid-summer to end of Autumn |
| Density | Thick |
| Growth Type | Clump-forming, herbaceous perennial |
| Height | Low, 30-60 cm |
| Width | Medium |
| Leaf | |
| Color | 137C |
| Characteristics | Basal, linear, tapered, falcate |
| Mean Length (mm) | 584 |
| Mean Width (mm) | 17.5 |
| Apex | Pointed |
| Margin | Smooth |
| Texture | Slightly ribbed with prominent mid-vein |
| Shape | Deep concave to flat, lanceolate, |
| | upright bending back at top ⅓^{rd} |
| Petiole | Nil |
| Stem | Sessile |
| Scapes | |
| Branching arrangement | Taller than foliage, non branching |
| Surface texture | Smooth |
| Mean stem diameter (mm) | 5.7 |
| (just below inflorescence) | |
| Lenath (mm) | 635 to 689 |
| Perianth | |
| Shape | Campanulate |
| Lenticel | Nil |
| INFLORESCENCE | |
| Length of flowering period | Mid Summer to Late Autumn |
| Mean Number of florets | |
| Mean floret length (mm) | 54.7 |
| Shape | Trumpet |
| Mean head size diameter (mm) | 90.6 |
| Lastingness of bloom | 1 day |
| Fragrance of bloom | weak |
| Profile Upper | Flat |
| Profile Lower | V-Shaped |
| Color Midzone outside | 185A |
| Color Margin outside | 185A |
| Color Midzone inside | 185A |
| Color Margin inside | 185A |
| Flower bud | |
| Shape | Long Ovate |
| Mean Length (mm) | 45.5 |
| (at time of sepal separation) | |
| Mean width (mm) | 10.8 |
| (at time of sepal separation) | |
| Color | 1C with tinges of 185A |
| Petal | |
| Number | 6 |
| Mean length (mm) | Inner 51.0 |
| (as at separation) | Outer 49.7 |
| Mean width (mm) | Inner 29.0 |
| | Outer 92.2 |
| Arrangement | 6 uniting to form a narrow basal tube, |
| | free parts spreading with the inner |
| | three petals wider and ruffled |
| Pedicel | |
| Mean width (mm) | 3 |
| Color | 144B |
| Prickles | No |
| Mean Length (mm) | 6 |
| Sepals | Nil |
| Basal Spot | |
| Presence | Present |
| Color | 14B-150B |
| Stamen | |
| Filament color | 17A |
| Mean filament length (mm) | 4.5 |
| Anther color | first two-thirds: 12B |
| | last third: 51C |
| Mean anther length (mm) | 37.1 |
| (base to bend) | |
| Pistils | |
| Mean style length (mm) | 61.6 |
| Style color | First three-quarters is 12B |
| | Last quarter is 51C |

### General Characteristics

‘BERRUB’ is primarily distinguished from other cultivars of Daylily genus by its distinctive burgundy red flower color. ‘BERRUB’ is a perennial full compact plant with repeat blooming over a period from early summer until first frost. The foliage is in the form of long, medium green leaves that are slightly ribbed with a prominent mid-vein and reaching about 23 inches in length (584 mm) while the scapes reach about 25 to 27 inches (635-689 mm) in height to carry the flowers above the foliage.

1. A new and distinct variety of Daylily plant, substantially as shown and described.
